(19) United States
(12) Patent Application Publication
(10) Pub. No.: US 2002/0198230 A1
(43) Pub. Date: Dec. 26, 2002

(54) METHOD AND APPARATUS FOR MICROWAVE ASSISTED CHEMICAL REACTIONS

(76) Inventor: HOWARD M. KINGSTON, PITTSBURGH, PA (US)

Correspondence Address: ARNOLD B SILVERMAN, ECKERT SEAMANS CHERIN & MELLOTT, 600 GRANT STREET 44TH FLOOR, PITTSBURGH, PA, 15219

(21) Appl. No.: 09/262,597

(22) Filed: Mar. 4, 1999

Related U.S. Application Data
(63) Continuation-in-part of application No. 08/458,757, filed on Jun. 2, 1995, now Pat. No. 5,883,349.
Application No. 08/458,757 is a continuation-in-part of application No. 08/357,097, filed on Dec. 15, 1994, now abandoned.
Application No. 08/357,097 is a continuation of application No. 08/127,263, filed on Sep. 24, 1993, now abandoned.

(51) Int. Cl. A61K 31/47; A01N 43/42

(52) U.S. Cl. 514/310

(57) ABSTRACT

A method of microwave assisted chemical reaction includes providing a microwavable reaction vessel which contains at least one material in a sample. The sample is heated by microwave energy to elevate the temperature of the reagent and at least partially volatilize the sample to establish a gas phase within the vessel followed by positive cooling of the gas phase to reduce the temperature and responsively reduce the pressure of the gas phase without effecting substantial cooling of the liquid phase. The method may involve employing cooling exteriorly of and adjacent to the gas phase containing portion of the vessel or cooling by means of a coolant flowing within coils disposed in the interior of the vessel or both. The process is preferably a continuous process. The apparatus may be a vessel transparent to microwave energy for receiving the sample. The vessel has space overlying the liquid phase containing portion for a gas phase. Structures for cooling means for positively cooling the gas phase to reduce the pressure of the gas phase without effecting substantial cooling of the reagent are provided. These structures for cooling may be contained within the vessel, exteriorly of the vessel or modification of the vessel configuration to facilitate gas phase cooling or combinations thereof. The invention also provides a method and associated apparatus for employing microwave heating and cooling of gases evolving from the heated sample in performing chemical analysis of samples. The invention further contemplates using such a system to purify samples.

### CROSS-REFERENCE TO RELATED APPLICATIONS

This application is a continuation-in-part of U.S. patent application Ser. No. 08/458,757, filed Jun. 2, 1995, entitled “Method and Apparatus for Microwave Assisted Chemical Reactions,” which was a continuation-in-part of U.S. patent application Ser. No. 08/357,097, filed Dec. 15, 1994, entitled “Method and Apparatus for Microwave Assisted Chemical Reactions” which was a continuation of U.S. patent application Ser. No. 08/127,263, filed Sep. 24, 1993, entitled “Method and Apparatus for Microwave Assisted Chemical Reactions.”

### BACKGROUND OF THE INVENTION

1. Field of the Invention

This invention relates to a method of microwave assisted chemical reactions, such as sample preparation, synthesis, derivatization, extraction, chemical analysis and distillation purification which involves reduced pressure within the vessel and associated apparatus for accomplishing this objective.

2. Description of the Prior Art

The use of microwave energy in analytical chemistry to provide heat to assist chemical reactions has been known for approximately 15 years. See, generally, Mingos et al., Applications of Microwave Dielectric Heating Effects to Synthetic Problems in Chemistry, Chem. Soc. Rev. 1991, 20, pp. 1-47.

It has been known to employ such microwave heating in sample preparation. See, Kingston et al., Comparison of Microwave Versus Conventional Dissolution of Environmental Applications, Spectroscopy 7 (9) November/December 1992, pp. 20-27. One approach involves an open-vessel approach in which the result is achieved with the assist of microwave heating. An alternate approach is the so called “closed-vessel” microwave sample preparation.

It has been know to use microwave energy for various types of environmental processes. For example, microwave energy, such as that produced by a nominal or high intensity microwave oven, has been employed to extract pesticides from sediment samples. See, Onuska et al., Extraction of Pesticides from Sediments Using a Microwave Technique, Chromatographia, Vol. 36, pp. 191-194 (1993). Microwave heating has also been employed in effecting hydrolysis of proteins. See, Margolis et al., The Hydrolysis of Proteins by Microwave Energy, Journal of Automatic Chemistry, Vol. 13, No. 3, pp. 93-95 (May/June 1991).

It has also been known to employ microwave energy in a closed vessel digestion system wherein a closed Teflon PFA vessel has an organic sample, an inorganic sample or a combination subjected to acid decomposition under the influence of microwave energy. See, Kingston et al., Microwave Energy for Acid Decomposition at Elevated Temperatures and Pressures Using Biological and Botanical Samples, Anal. Chem., 58, pp. 2534-2541, (October, 1986).

In such closed vessel microwave sample preparation techniques, typically, one or more materials which will become the sample are mixed or dissolved in a suitable liquid reagent. The liquid reagent occupies a portion of the volume of the relatively small vessel and is subjected to chemical alteration under the influence of the microwave heating, thereby creating a gas phase in addition to the liquid phase within the vessel. The microwave heating results in increased temperatures and pressures within the vessel which can present a potential safety hazard through vessel failure. The increased temperature is required for advancement of the reaction rate, but the pressure is a property of the heat flow characteristics of the vessel and microwave interaction.

It has been known to control heat loss from the vessel by providing a jacket of thermal insulation around the vessel which also acts to strengthen the vessel. See, generally, Mingos et al., Applications of Microwave Dielectric Heating Effects to Synthetic Problems in Chemistry, Chem. Soc. Rev., 1991, 20, pp.1-47 and Chapter 6, Introduction to Microwave Sample Preparation Theory and Practice by Kingston et al., American Chemical Society, 1988, pp.93-154.

U.S. Pat. No. 5,215,715 discloses a method of digesting materials which are dispersed in an acid digesting medium, which dispersion is subjected to microwave heating in a first chamber and then both the gas and liquid phases of the dispersion are cooled in another chamber. There is no segregated cooling of the gas phase while heating the liquid phase. There is also no recognition of the pressure relationship between the gas phase and liquid phase during microwave radiation.

In prior art practices, pressure within the vessel has been permitted to form at whatever natural level occurred due to the specific reagents, temperature, reaction products, microwave interaction and heat flow of the vessel.

There remains, therefore, a very real and substantial need for a more efficient and safe means of microwave sample preparation in a closed vessel.

### SUMMARY OF THE INVENTION

The present invention has solved the above-described problems by providing a method and apparatus wherein a microwavable reaction vessel is provided with a liquid reagent mixture and/or sample. For convenience of reference herein, both of these categories and any similar materials to be processed will be referred to as a “sample. ” The sample is heated so as to elevate the temperature thereof to establish at least partial volatization of the sample and thereby create a gas phase overlying the liquid reagent within the vessel. The gas phase is positively cooled to reduce the temperature in the gas phase and, responsive to said temperature reduction, reducing the pressure without effecting substantial cooling of the liquid reagent.

The cooling of the gas phase may be effected by providing channels for coolant flow exteriorly of the vessel or coolant flow within the vessel within coils or both. In this manner, the temperature and pressure of the gas phase are reduced in the preferred practice of the invention, while the coolant flowing in the cooling conduits, whether they are disposed interiorly or exteriorly of the vessel or both, does not directly cool the liquid reagent.

The apparatus for practicing the method preferably includes a vessel, such as a vessel or vessel liner made from a suitable polymer or fluoropolymer, such as polytetrafluoroethylene, TFM or perfluoroalkoxy, which is transparent to microwave energy and receives the liquid reagent mixture and/or sample. The vessel may also utilize an outer casing of a different material, such as polyetherimide, glass filled polyetherimide, and other suitable materials. The vessel has additional capacity for the gas phase. Cooling means provide for positive cooling of the gas phase to reduce the temperature and pressure of the gas phase. The cooling means has passageways for the flow of coolant. The passageways may be disposed exteriorly of the vessel and adjacent to the outer walls of the vessel with the passageways not being disposed adjacent to the sample or liquid reagent containing portion of the vessel. In another embodiment, the passageways are coils disposed within the gas phase portion of the vessel.

The invention in another embodiment also provides microwave assisted chemical analysis wherein a sample within a vessel is subjected to heating by microwave energy to volatilize at least a portion of the sample to establish a gas phase with the gas phase being cooled while the heating of the sample is continued. Subsequently, the unvolatilized portion of the sample is analyzed to determine the molecular and/or elemental components present therein. Also, if desired, the volatilized portion of the sample may be analyzed. In one embodiment, silicon such as that used in computer chips is analyzed to determine the identity and quantity of trace elements present therein.

In another embodiment of the invention, the method is employed to purify a portion of a sample. The procedure may be basically that employed for analysis with distillation resulting in purification.

The vessel has portions transparent to microwave energy so that the sample contained therein may be heated to volatilize portions thereof. The gas phase is cooled simultaneously with the heating through microwave energy of the sample. The distillation components of the sample may be purified.

It is an object of the present invention to provide a method and apparatus for closed vessel microwave assisted chemical reactions which effectively reduces the pressure in the gas phase within the vessel.

It is another object of the invention to provide such a system wherein the pressure reduction in the gas phase is effected through positive cooling to reduce the temperature thereof.

It is another object of the present invention to provide such a system which may be employed in microwave digestion and reaction bombs.

It is a further object of the present invention to provide such a system which is employed in preparing chemical samples for later analysis.

It is a further object of the invention which permits microwave heating of the sample to elevate its temperature simultaneous with positive cooling of the gas phase.

It is yet another object of the invention to provide such a system which is adapted to accomplish sample preparation in a much more rapid manner than those previously known.

It is yet another object of the present invention to provide a system which facilitates microwave heating of samples for purposes of chemical analysis thereof.

It is yet another object of the present invention to provide such a system wherein the molecular and/or elemental components in samples can be separated through the simultaneous sample heating through microwave energy while cooling of gaseous components generated by such heating and subsequently chemically analyzed.

It is yet another object of the present invention to employ such a system wherein samples can be purified through distillation created by microwave heating.

It is a further object of the present invention to provide such a system which will contribute to increased durability of the vessels.

These and other objects of the invention will be more fully understood from the following description of the invention on reference to the illustrations appended hereto.

### BRIEF DESCRIPTION OF THE DRAWINGS

FIG. 1 is a schematic cross-sectional illustration of a form of vessel of the present invention having external cooling passageways.

FIG. 2 is a schematic cross-sectional illustration of a form of vessel of the present invention having internal cooling passageways.

FIG. 3 is a schematic flow chart showing a continuous system of the present invention.

FIG. 4 is a comparison of reaction conditions in Teflon and insulated vessels.

FIG. 5 is a partially schematic illustration of apparatus employable in the chemical analysis of a sample in accordance with the present invention.

FIG. 6 is a partially schematic illustration of apparatus suitable for use of chemical analysis in the present invention showing the sample after partial volatilization.

FIG. 7 is a plot of time versus solution temperature and liner temperature.

FIG. 8 is a partially schematic illustration of an embodiment of the invention usable in connection with chemical analysis or purification in accordance with the present invention.

FIG. 9 is similar to FIG. 8, but showing a modified form of cooling apparatus.

### DESCRIPTION OF THE PREFERRED EMBODIMENTS

Microwave vessels employed in chemical reactions, such as sample preparation, synthesis, derivatization and extraction generally are of relatively moderate size and may have an interior volume of about 1 mL to 500 mL and preferably in the range of about 1 mL to 125 mL. The vessels may have any desired configuration, but are frequently generally cylindrical in shape. They may be made of Teflon (tetrafluoroethylene, PFA or TFM or PTFE) or other fluorinated carbon plastics with a removable lid adapted to seal in place as by threaded or pressure fitted securement to maintain the desired amount of pressure, which for this type vessel, might be in the order of up to about 10 atmospheres. Another type of vessel would have a plastic casing for rigidness and pressure stability with a Teflon, plastic or quartz liner for chemical inertness and be adapted to withstand pressures of about 5 to 20 atmospheres. In this latter category, the vessel may be designed so as to withstand pressures of 40 to 100 atmospheres.

Closed vessel digestion will generally achieve higher temperatures because the boiling point of the reagent is raised by the pressure produced within the vessel. The higher temperature in the closed vessel will, however, greatly reduce the time required for reaction. The closed vessel also resists evaporation and there is, therefore, no need to add reagent to maintain the desired volume.

The vessels are effectively transparent to microwave energy so as to permit them to be introduced into a microwave oven and the reagents and samples contained in them to be heated to the desired temperature. As the liquid reagent containing one or more materials is heated, a gas phase is formed through the vaporization of the solvent and/or the chemical materials. The sample or samples will generally be mixed with a liquid reagent which may, for example, be nitric acid employed in microwave-heated digestions. In order to maintain pressure levels within the desired ranges of safety and contribute to durability of the vessels, as well as achieving the desired temperature which is most beneficial for the chemical reaction contemplated, the present invention provides positive cooling to the gas phase contained within the vessel while resisting effecting meaningful cooling of the liquid reagent.

For a given liquid reagent, the absorption of microwave energy can be calculated at a specific frequency employing Equation 1.

$$Pabsorbed = \frac{{KC}_{p} m Δ T}{t} \qquad ( 1 )$$

wherein:

P=is the apparent power absorbed by the sample in watts (W),

(W =joules/sec);

K is the conversion factor for thermochemical calories/sec to W, which is 4.184;

Cₚ=is the heat capacity, thermal capacity, or specific heat (cal./g.ΔC);

m=is the mass of the sample in grams (g);

ΔT=is T_{f}, the final temperature minus Tᵢ, the initial temperature (AC); and

t=is the time in seconds (s).

$$\begin{matrix}{T}_{f} = {T}_{i} + \frac{Pabsorbed \cdot t}{K \cdot {C}_{p} \cdot m} & ( 2 ) \\ {T}_{f} = {T}_{i} + \frac{Pabsorbed \cdot t}{K \cdot {C}_{p} \cdot m} - HeatLoss & ( 3 )\end{matrix}$$

In the event that no energy is permitted to escape from the vessel, the final temperature can be determined by equation 2.

As shown in Equation 3, a lower temperature is achieved if energy is permitted to escape. This escape can be primarily from the gas phase as it has the greatest area of cool vessel wall to contact.

In the present invention, active cooling of the gas phase serves to reduce the gas phase pressure. If desired, the microwave energy applied to the liquid phase sample may be increased to compensate for the thermal energy losses to the gas phase.

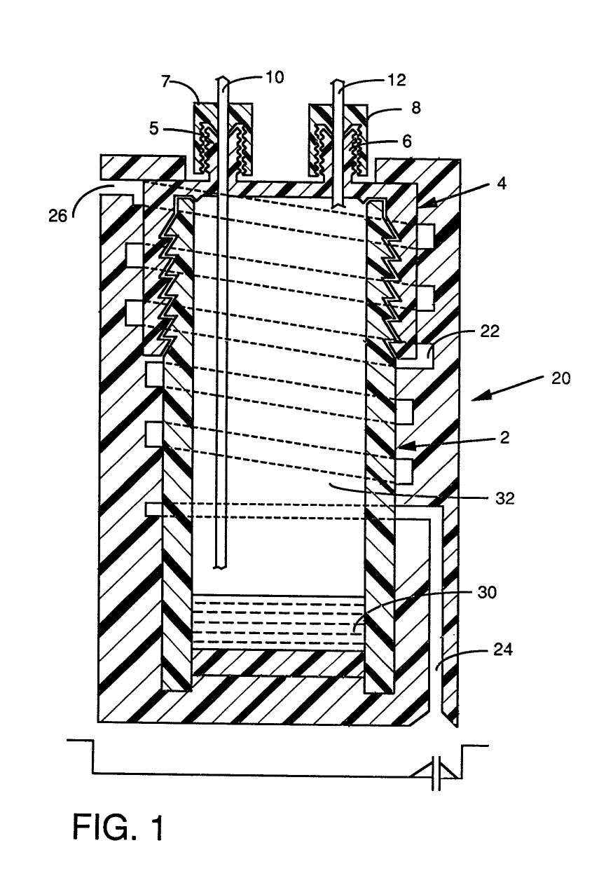

Referring now more specifically to FIG. 1 wherein there is shown a closed microwave reaction vessel which may be adapted for use with automation or a robot as distinguished from individual human handling, if desired. There is shown a vessel consisting of a liner 2 which may be composed of a suitable fluorinated carbon plastic, such as tetrafluoroethylene which is sold under the trade designation “Teflon” or other material having suitable strength, microwave transparency, and chemical inertness. The vessel liner 2 has a threaded closure 4 intimately secured in sealing relationship to the liner 2. The closure 4, in the form shown, has a pair of upwardly projecting, threadedly secured port defining members 5, 6 to which apertured closures 7, 8, respectively, are secured. While these port closures 7, 8 may be closed off if desired, in the illustrated embodiment temperature probes 10, 12, respectively, extend into the vessel 2 to different depths. These probes 10, 12 may be of any conventional type and are sealingly secured to the port closure 5, 7, 8.

Positioned in surrounding relationship with respect to liner 2 is an outer wall or casement 20 which is in intimate surface-to-surface contact with the exterior of the vessel 2 and closure 4. The casement 20 may be provided in multiple pieces (not shown) assembled around the vessel by any desired means known to those skilled in the art. The vessel 2, closure 4, and outer wall 20 are preferably of generally cylindrical configuration. The outer wall or casing 20 has an inwardly open continuous helical groove 22 which cooperates with exterior of the vessel liner 2 and closure 4 to create a continuous coolant flow passageway. The passageway is spaced (measured along the vessel longitudinal axis) from the sample liquid reagent received portion 30 of the vessel. A coolant entry channel 24 is defined within casement 20 and is in communication with passageway 22. Coolant is discharged through exit channel 26. The coolant will preferably be captured as it emerges from channel 26 and subjected to a heat exchanging temperature reduction after which it may be reintroduced into coolant entry channel for another cycle of operation. The coolant may be microwave non-absorbing, moderately absorbing, or strongly absorbing material that may be in a gas or a liquid phase.

If desired, the coolant passageways may be provided in other ways. For example, such as by a single ring, which is inwardly open to provide an annular passageway in cooperation with or adjacent to the exterior of the vessel. Also, an axially elongated single ring or a plurality of such rings either interconnected or individually supplied with coolant may be employed.

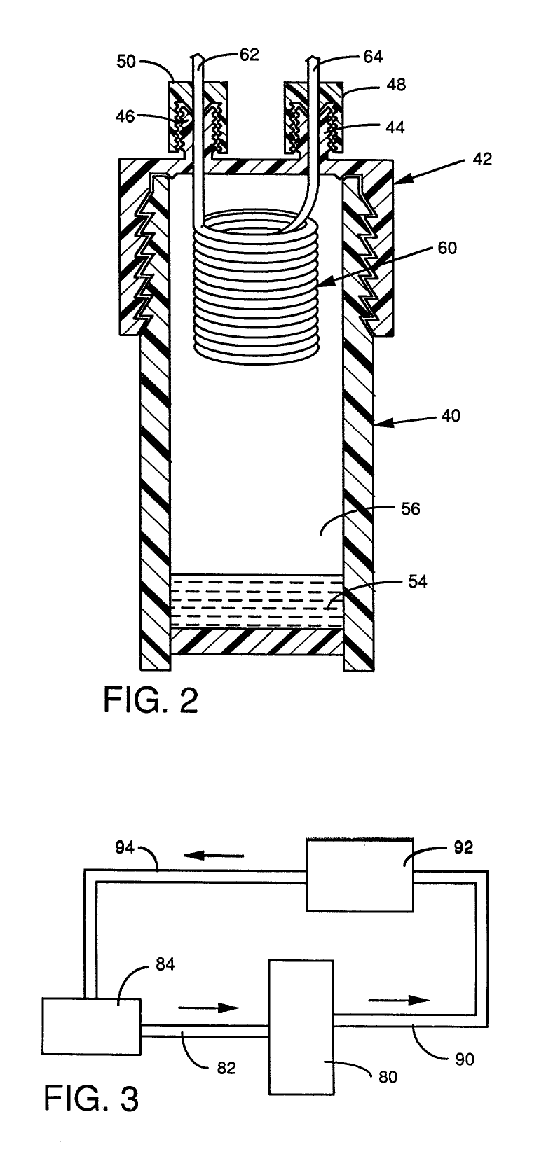

Referring now to FIG. 2 in greater detail there is shown a microwavable vessel 40 having threadedly and sealingly secured thereto a closure 42 which has a pair of externally threaded ports 44, 46 to which are secured threaded sealing closures 48, 50 respectively. The liquid reagent mixture or sample 54 is contained within the lower portion of the vessel interior and the gas phase 56 appears thereabove. A coolant coil 60 is received within the interior vessel 40 and has an entry end 62 and a discharge end 64. In effecting cooling of the gas phase 56 without effecting substantial cooling of the liquid reagent mixture 54, coolant is permitted to flow into entry 62, assume a heat exchanging interaction with the gas phase and then emerge at an elevated temperature at discharge end 64. The coolant coming out of end 64 is subsequently subjected to a heat exchanging process wherein the temperature of the coolant is reduced after which the coolant is reintroduced through entry 62. It will be appreciated that, in this manner, continuous cooling of the gas phase will be effected to thereby reduce the pressure within the gas phase 56. If desired, coils of additional length or multiple coils having separate entries may be employed. If desired, radiator structures may be employed in the vessel interior in lieu of the coil or coils.

It will be appreciated that the embodiment shown in FIGS. 1 and 2 are not mutually exclusive and that the coil or coils employed in connection with the embodiment of FIG. 2 may be employed in addition to the passageway containing outer wall 24 of FIG. 1 in order to achieve the desired degree of temperature reduction of the gas phase and corresponding reduction of pressure in the vessel interior.

The partial traditional equilibrium pressures and the partial pressures of the reagents and sample and reaction byproducts do not hold in this system as equilibrium of temperature between liquid and gas phases is never reached. Condensation of several components may occur reducing the partial pressure of one or more thus reducing the total pressure in the vessel. A dynamic non-equilibrium condition is established that is unique to microwave reagent closed vessel systems such as these and is a new relationship that is being employed to produce these new reaction conditions.

Referring now to FIG. 3, there is shown schematically a block diagram of a continuous or semi-continuous flow system of the present invention. The gas phase portion of vessel 80 receives coolant through pipe 82 by means of pump 84. After the coolant absorbs heat from the gas phase contained within vessel 80, the elevated temperature coolant emerges through pipe 90 and enters heat exchanger 92 wherein heat is withdrawn and the coolant is reduced to a temperature desired for introduction into the gas portion of vessel 80. The reduced temperature coolant emerges from the heat exchanger 92 and is carried by pipe 94 to pump 84 for reintroduction into vessel 80.

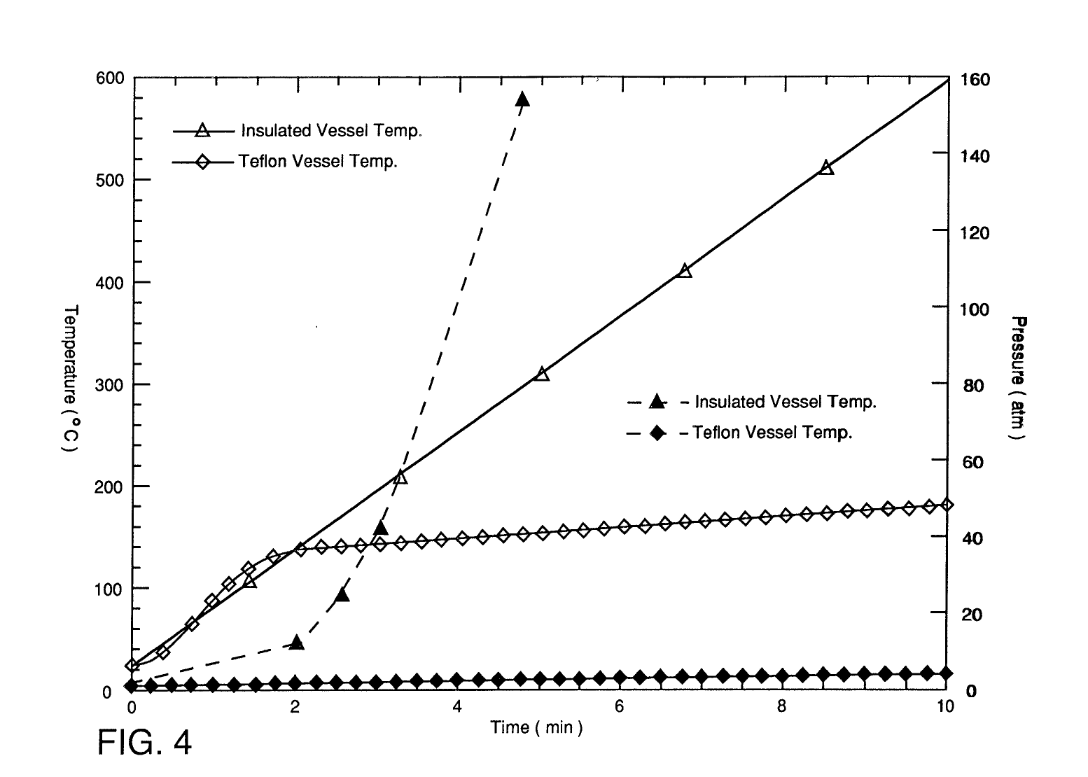

Referring to FIG. 4, there is shown a plot of temperature in degrees centigrade and pressure in atmospheres as related to time. It compares a thermally insulated vessel with a thermally uninsulated vessel, i.e., a Teflon vessel. The difference in pressure inside the vessels is due to the loss of thermal energy in the gas phase. For example, the pressure of 6* 10 mL of concentrated nitric acid irradiated at 574 watts for 10 minutes at 180° C. is about 40 psi in the insulated vessel and is only about 8 psi in the uninsulated vessel. The absorption of microwave energy which can be calculated from equation 1 is the same for a given liquid.

### EXAMPLE 1

In order to enhance the understanding of the invention, an example will be provided. A closed microwave vessel having an interior volume of 120 mL is provided with 20 mL of nitric acid mixed with a 0.5 gram liver tissue (material) in a closed vessel acid digestion process. The vessel was exposed to 500 watts of microwave energy for a period of 10 minutes to establish a liquid temperature of 190° C. and a liquid partial pressure inside the vessel of 620 psi without cooling. When a similar situation is constructed with cooling of the gas phase, there was established a pressure with the acid and digestion products of 120 psi inside the vessel. This demonstrates positive cooling by a method of the present invention employing a method of air coolant to produce after 10 minutes a gas phase temperature of 130° C. and a gas phase partial pressure of 120 psi without effecting a substantial reduction in the liquid phase temperature. A 650 watt power was applied in the second example to maintain the liquid temperature at 190° C. As a result, the acid digestion was effected while reducing the vessel pressure by 500 psi.

The coolant may be a gas or liquid with or without entrained solids, and is preferably transparent to microwave energy. Among the preferred coolant, materials are one or more materials selected from the group consisting of air, CO₂, freon, gaseous N₂ and liquid N₂.

The system of the present invention builds upon and enhances certain scientific principles as applied to solve a particular problem. The unique nature of microwave interaction and two distinct heat transfer mechanisms permits the cooling of the gas phase while continuing to heat the liquid phase. Heating a liquid in a microwave field is commonly referred to as dielectric loss. The two primary mechanisms are dipole rotation and ionic conduction. See, generally, Kingston, H. M. and Jassle, L. B., Eds., “Introduction to Microwave Sample Preparation: Theory and Practice,” ACS Professional Reference Book, American Chemical Society, Washington, D.C., 1988, pp. 9-15. Ionic conduction is the conductive migration of dissolved ions in the applied electromagnetic field. Dipole rotation is the alignment, due to the electric field, of molecules that have permanent or induced dipole moments. When a molecule vaporizes and is converted to the gas phase, from the liquid phase, charged ions are left in the liquid phase, thereby eliminating this heating mechanism. In addition, rotation of the molecule in the gas phase does not efficiently transfer heat, as rotation without collision, does not add heat to the gas phase. Gas molecules frequently collide with the surfaces of the vessel. These surfaces are not heated by microwave energy and are actively cooled, thereby cooling the gas phase. The vessel is generally made of a material which is usually essentially microwave transparent. The gas phase is not efficiently heated by the microwave field even though the gas phase and liquid phase both exist in the same microwave field. These heating conditions are unique to the microwave environment. The present invention employs the ability to cool the gas phase while continuing to heat the liquid phase in this environment. The present invention involves intentionally cooling the gas phase while heating the liquid phase to effect the reduction of the internal vessel pressure while maintaining a relatively high liquid temperature in which various chemical reactions are conducted.

It will be appreciated, therefore, that the present invention provides a method and apparatus for pressure control and reduction in microwave-assisted chemical reaction systems. This is accomplished through positive cooling of the gas phase which is in contact with the liquid phase in the chemical reaction vessels without effecting significant reduction in temperature of the liquid phase. The positive cooling of the gas phase facilitates corresponding pressure control of the gas phase in order to achieve the desired chemical or physical parameters during and following the reaction period. The reactions in the liquid phase can, therefore, be carried out without undesired interference as a result of the positive cooling of the gas phase. The practice of the present invention will generally reduce the pressure in the gas phase about 50 to 95 percent and preferably about 60 to 90 percent. If desired, positive cooling action may be terminated or regulated when the desired gas phase pressure has been attained.

It will be appreciated that the present invention permits efficient thermally activated chemical reactions to occur at the desired temperature, while facilitating a reduction in pressure within the vessel at that temperature. This facilitates improved process efficiency, safety and durability. Improvement of the durability of the vessel is achieved through maintaining the integrity by resisting overheating of the casing in double walled vessels. Also, in the embodiment of FIG. 1, the coolant may serve to carry away sample or reaction products that might become trapped between the outer wall 20 and the vessel liner 2.

Also, if desired, the vessel might be formed with partially hollow outwardly projecting fins or ribs to facilitate radiation loss of heat from the gas phase. In the alternative, multi-walled vent openings may be provided in the outer wall to enhance cooling of the gas phase.

A plurality of circumferentially spaced, axially oriented ribs may be provided within the gas phase region of the vessel, but not in the liquid phase portion, such a construction will be deemed positive cooling within the context of the present invention.

In addition to the foregoing the turntable onto which the vessel is placed may be cooled. The hollow turntable top might have a recess which receives an upper portion of the vessel in intimate contact therewith. Coolant may be circulated within the hollow turntable top.

While not the preferred practice of the invention, if desired, gas may be withdrawn from the gas phase of the vessel, cooled and subsequently returned to the gas phase of the vessel.

The vessel, for example, may be a container that holds volumes from about 50 mL to 500 mL or may be an elongated tube which is closed to the atmosphere and in which the sample flows through the microwave field.

An elongated tube may have the sample and gas phase moving by the microwave source and cooling means so as to permit both heating of the sample and cooling of the gas phase which would be present in the sealed tube. As this embodiment would involve commingling of the liquid sample and gas phase, it is not the preferred embodiment.

Two additional embodiments of the invention wherein the method and apparatus are employed in chemical analysis of a specimen or purification thereof will be considered.

The sample may include solvents and analytes of interest with the analytes of interest being (a) molecules, (b) elements, or (c) species of such molecules or elements. The microwave heating of the invention will create a gas phase through volatilization of a portion of the sample and an unvolatilized residue.

A fundamental review of microwave-enhanced chemistry has been presented recently that describes the common uses of microwave-enhanced chemistry in analytical chemistry and other microwave-enhanced chemical applications primarily in the laboratory. Descriptions of the fundamental heating and cooling mechanisms are described in Walter, P. J. et al., A Review of Overview of Microwave Assisted Sample Preparation”, Chapter 2, in Microwave Enhanced Chemistry: Fundamentals, Sample Preparation, and Applications, ACS Professional Reference Book Series, American Chemical Society, Washington, D.C., 1997; Kingston, H. M. et al., Microwave Enhanced Chemistry: Fundamentals, Sample Preparation, and Applications, ACS Professional Reference Book Series, American Chemical Society, Washington, D.C., 1997; Kingston, H. M. et al., “Environmental Microwave Sample Preparation: Fundamentals, Methods and Applications”, Chapter 3, in Microwave Enhanced Chemistry: Fundamentals. Sample Preparation, and Applications, ACS Professional Reference Book Series, American Chemical Society, Washington, D.C., 1997.

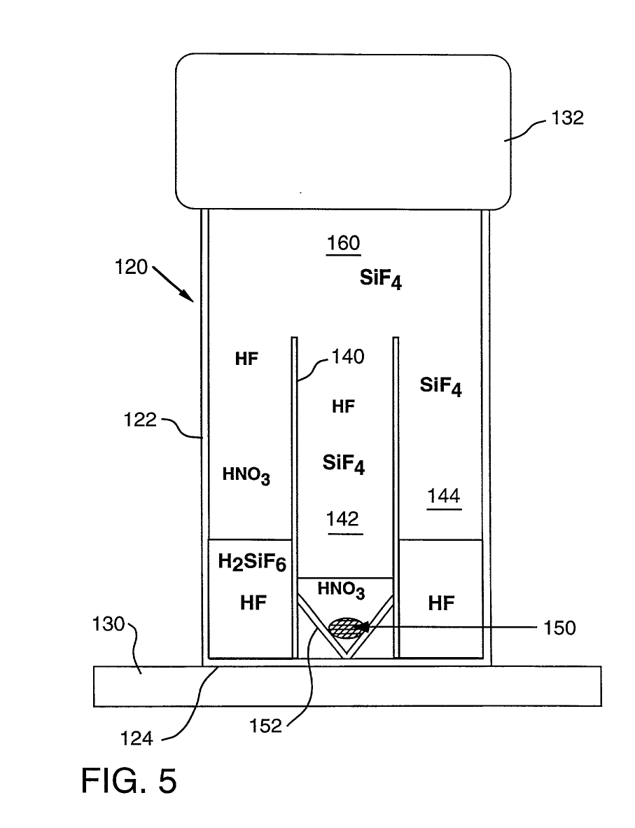

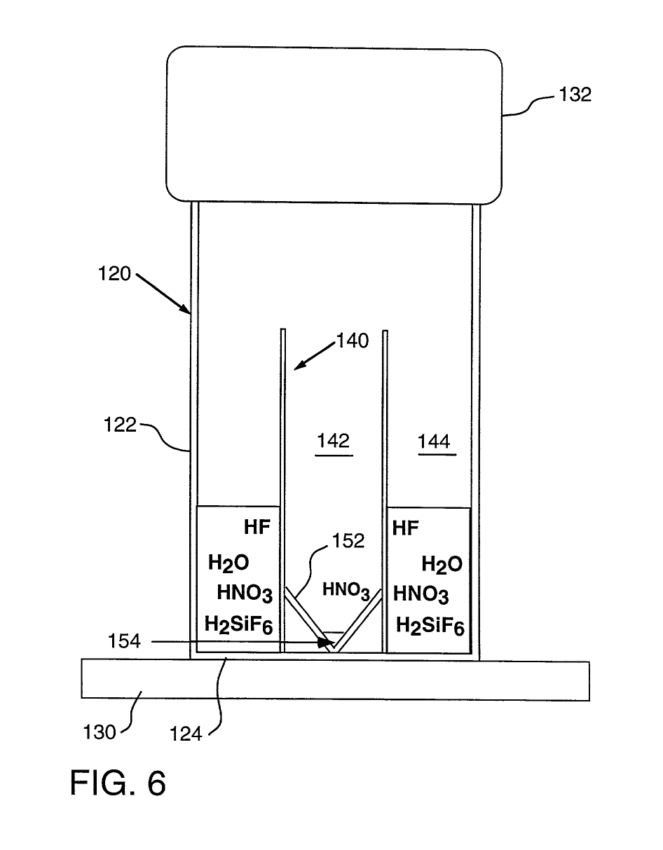

Referring to FIG. 5, a vessel 120 suitable for use in practicing another embodiment of the invention will be considered. In the form illustrated in FIGS. 5 and 6, use of the system of the invention for analytical purposes is contemplated. The vessel preferably has an annular sidewall 122 which may be generally cylindrical in shape and be composed a material transparent to microwave energy, such as a fluoropolymer. The container 120 has, in the form shown, an integrally formed base 124 which rests on a support 130 which may be made of any suitable thermally insulative resinous plastic, such as a fluoropolymer or polypropylene, for example. Sealingly secured to the upper portion of the wall 122 is a cooling section 132 which serves to reduce the temperature of the vapor phase. The cooling section 132 may be removable to permit access to the interior of the container 120 for removal of residue, cleaning or other purposes.

In this embodiment of the invention, with reference to FIG. 5, the vessel 120 is subdivided by continuous container 140 into a first compartment 142 which may be generally cylindrical and an annular second compartment 144 which is positioned therearound. The container 140 has a lesser height than exterior wall 122. In this embodiment, the sample 150 is supported on interior base 152 within compartment 142. Nitric acid (HNO₃) is contained within compartment 142 and hydrofluoric acid (HF) is contained within compartment 144. As the compartments 142, 144 are in communication with each other, when the microwave energy serves to volatilize the nitric acid and hydrofluoric acid, in a manner to be described hereinafter, gas is evolved and moves upward into region 160 and the trace elements 154 remain in compartment 142 on base 152 (FIG. 6). Assuming that the sample 152 was silicon such as might be used in computer chips, one could then determine the purity of the silicon by identifying and quantifying the remaining trace elements by any suitable means.

### EXAMPLE 2

In this example the cooling of the gas phase and evaporation of molecular components of the sample while heating of the liquid phase provide for a unique chemical processing environment. The target sample in this example is polycrystalline silicon. This material is used for the manufacture of integrated circuits and computer chips. The analysis of trace elements in this material may be critical to the application and use of this material.

One problem in the analysis of this material is that once dissolved in acids, the matrix, in the form of silicon oxides and or fluorides, remains with trace elements in solution. This increases the salt content or solids content of the final solution to a point that analysis by some instruments directly is difficult. For example, the use of inductively coupled plasma mass spectrometry (ICP-MS) for analysis is hampered by the clogging of the sample cones due to the buildup of silicon salts or oxides during aspiration of solutions of acid dissolved polycrystalline silicon in water diluted acid solutions. This problem is addressed by this embodiment of the invention. The general approach is applicable in other applications that would also benefit from matrix modifications during decomposition and alterations of chemistry based on mechanisms of phase energy distribution and control.

Referring to FIG. 5, a sample 150, which may be polycrystalline silicon, is placed in HNO₃ in the inner compartment 142 and HF is placed in the outer compartment 144. The HF is distilled into the inner compartment 142 and SiF₄ is distilled to the outer compartment 144. The outer surface of the vessel is cooled with a fan (not shown) pulling cool air at about 20° C. over the outer surface of the vessel 120 while the bottom wall 124 of the vessel 120 is in contact with an enlarged polypropylene plate 130 to resist airflow. Alternatively, additional thermal shielding can be placed around the vessel 120 to the level of the acid in the outer compartment 144. Additional cooling through cooling unit 132 and/or active removal of the gas phase or addition of additional cool air may be employed, but is not necessary to accomplish the desired effect.

Originally, 5 mL or 7.3 g of concentrated nitric acid HNO₃ is added to the inner compartment 142. Originally, 0.5 g of silicon is added to the inner compartment 142. The annular outer compartment 144 is approximately 100 to 150 mL in volume and the inner compartment 144 is approximately 10 to 20 mL in volume. Both are made from a relatively microwave transparent material such as a fluoropolymer. They are also insulating materials such as a fluoropolymer. The open tops in compartments 142, 144 are open sufficiently to permit gas to pass between the compartments 142, 144. The liquid phase is directly heated and partially thermally insulated from cooling by the bottom plate 130. Originally, 10 mL or 11.7 g of concentrated HF is added to the outer vessel. Based on a collection of fundamental chemical laws and principles such as Henry's law (the amount of a gas dissolved in a liquid is proportional to the partial pressure of the gas above the liquid) and the fundamental heating of the liquid phase by the microwave energy and its relative inability to heat the gas phase; and by cooling of the gas phase in contact with the cooled vessel wall or cooling unit a quantity of the HF is dissolved in the nitric acid and begins the reaction with the silicon and trace elements as described in the follows chemical expressions.

3Si+4HNO₃+12HF→3SiF₄↑+4NO+8 H₂O (1)

Si+4HNO₃+6HF→H₂SiF₆+4NO₂↑+H₂O (2)

A sufficient quantity of HF in the outer compartment 144 facilitates the SiF₄ being dissolved therein and also distills HF to the inner compartment 142 to complete the reaction as represented by reaction (3).

In the outer compartment 144 an additional reaction occurs and assists with the transfer of the silicone fluoride. Silicon fluoride reacts with the hydrofluoric acid to form fluorosilic acid.

SiF₄(g)+2HF→H₂SiF₆ (3)

If sufficient water is present in the hydrofluoric acid additional reactions can occur such as described in equations 4 and 5 which produce silica gel and also assist in removing molecular forms of silicon from the inner vessel.

H₂SiF₆+4H₂O<==>H₄SiO₄+6HF (4)

3SiF₄+4H₂O→2H₂SiF₆+H₄SiO₄(silica gel) (5)

The specific quantities of these reagents and the sample which are needed to perform these reactions properly and efficiently can readily be determined. If the HF to Si ratio is above 6:1 then these reactions are as set forth. Below 4:1 ratio HF to Si, there will tend to be residual unreacted silicon. The final conditions in the inner compartment 142 result in less than 0.4 g of combined nitric acid and hydrofluoric acid remaining with less than 0.0005 g residual Si remaining in the inner compartment 142. The solution has been found to contain less than 250 μg of Si. SiF₄ (tetrafluorosilane or silicon tetrafluoride ) is a gas with a boiling point of −86° C. and is soluble in hydrofluoric acid. The solution remaining in the inner compartment 142 may be diluted and the trace elements directly determined by may analytical analysis methods known to those skilled in the art, such as ICP-MS, or X-ray fluorescence, for example. Each decomposition and combined distillation takes approximately 70 min. The conditions inside the vessel start at ambient and reach 240° C. maximum and 35 atm.

Losses of trace elements from the inner compartment 142 for elements in ionic form were not observed. The absence of ionic elemental losses from the inner compartment 142 has been tested by adding known quantities of key specific trace elements of interest in analysis by silicon producers such as Cr, Cu, Fe, Ni, and Zn. Specifically in these validation experiments 25 ng of each of these elements were added to the inner compartment 142 in acid solutions. At the 95% confidence level agreement that these elements were contained in the remaining solution in the inner compartment 142 and that they were absent in the remaining solution in the outer vessel was demonstrated using ICP-MS analysis. Table 1. provides data supporting the recovery study used in this example.

TABLE 1
(Uncertainties expressed in percent relative standard deviation) (RSD)

| Element | Recovery after procedure | % RSD |
|---|---|---|
| Cu | 98.4 | 7.2 |
| Co | 96.3 | 3.3 |
| Ni | 101.6 | 10.2 |
| Zn | 105.4 | 10.2 |

The inner compartment 142 remains cool above the acid level and is not heated by microwave radiation. The upper area 160 above the liquid level remains cool and is cooler than the liquid phase at the bottom. If this configuration were assembled in a thermal oven where the outer walls of both the inner and outer compartments 142, 144 would reach thermal equilibrium with the solution temperature this process would not be effectively carried out. In addition, the small amount of liquid is isolated from the thermal load of the inner compartment 142 solution as it evaporates to dryness and lowers the temperature as described in more detail in the next example. This configuration may have additional cooling added by means of cooling finger or coil or additional outer compartment or inner compartment wall cooling may be employed.

If the common practice of adding both nitric and hydrofluoric acid to the silicon were used, the sample would contain almost all the silicon and the analysis would be made very difficult due to the composition of high solids content.

### EXAMPLE 3

For the past 70 years it has been known in analytical chemistry that elements such as Sb, As, Hg, Sn, Se, Cr are volatilized from acid solutions especially hydrochloric acid. See, Applied Inorganic Analysis: with Special Reference to the Analysis of Metals, Minerals, and Rocks, by Hillebrand, Lundell, Bright, and Hoffman, 2^{nd} ed. John Wiley & Sons, Inc., NY, N.Y. 1953.Pgs 57-58, 78, 210, 259, 273, 285 & 297. A compendium of the last 60 years of inorganic acid analytical volatilization has been compiled. See, Walter, P. J. et al., A Review of Overview of Microwave Assisted Sample Preparation”, Chapter 2, in Microwave Enhanced Chemistry: Fundamentals, Sample Preparation and Applications, ACS Professional Reference Book Series, American Chemical Society, Washington, D.C., 1997; Kingston, H. M. et al., Microwave Enhanced Chemistry: Fundamentals, Sample Preparation and Applications, ACS Professional Reference Book Series, American Chemical Society, Washington, D.C., 1997; Kingston, H. M. et al., “Environmental Microwave Sample Preparation: Fundamentals, Methods and Applications”, Chapter 3, in Microwave Enhanced Chemistry: Fundamentals, Sample Preparation, and Applications, ACS Professional Reference Book Series, American Chemical Society, Washington, D.C., 1997. The volatility of many elements in hydrochloric acid is also known.

The need to evaporate and remove or exchange reagents such as acid and organic solvents occurs frequently. For example, many elements are not soluble as fluorides. Hydrofluoric acid however is an acid that effectively performs the opening out reaction on silicon dioxide the major matrix component in many rocks, soils, glass and ores. Hydrofluoric acid is easily evaporated using nitric acid and hydrochloric acids. These acids, especially nitric acid, produce aqueous soluble salts that are able to be aspirated into instruments such as atomic absorption and inductively coupled plasma optical emission and mass spectrometers as homogeneous solutions. Losses at this point have been a problem for many decades and many papers document the significance with minor alterations in solution and mechanical heating methods. See, Walter, P. J. et al., A Review of Overview of Microwave Assisted Sample Preparation”, Chapter 2, in Microwave Enhanced Chemistry: Fundamentals, Sample Preparation. and Applications, ACS Professional Reference Book Series, American Chemical Society, Washington, D.C., 1997.

The physical heating mechanism plays a meaningful role in this traditional evaporation problem. Boiling of these solutions physically removes elements as aerosols in droplets from bursting bubbles at the surface of the liquid. Some molecular forms of elemental compounds are volatile at elevated temperatures that are common on hot-plate surfaces or in flames. Many elemental molecular chlorides are volatile and at a few hundred degrees Centigrade have a significant vapor pressure. Up until the last two decades the only mechanisms available have been convection and conduction. In these mechanisms the contact of a hot plate or flame has been directly on the vessel, such as a glass beaker or flask for example. In convection and conduction as the acid, solvent and/or water evaporated they kept the temperature at or below the boiling point of the aziotropic mixture of the solution. As the solution went to dryness, however, the vessel achieved the maximum temperature close to that of the surface of the hot-plate or the flame which is some times several hundred degrees above the liquid boiling point. At dryness the temperature of the salts and molecular residue reach several hundred degrees. Direct volatilization of the molecular or metallic compounds or salts produced losses from the residue under these conditions. As the options available were only convection and conduction, there were not significant ways to overcome these mechanical disadvantages except to take the heat source away just as the sample approached dryness. This approach is not readily reproducible. Also sand and water baths were used to keep the temperatures low at dryness, however, these are very inefficient and require long periods of times.

The direct induction of energy using microwave frequencies has provided a new tool. It facilitates the use of solvent evaporation to preserve analyte sample components. Prior art microwave energy induction mechanisms are very different from previous convection and conduction methods. The changes in sample heating mechanisms and use of these differences permits exploitation in the evaporation applications. Gasses do not heat as liquids.

The present invention facilitates cooling the gas phase while heating the liquid phase in the same vessel. Additionally the energy absorption rate is not constant with geometry and mass. As the molecular target becomes small as compared to the microwave wavelength, the electromagnetic energy does not couple as efficiently and a cooling effect relative to the liquid occurs. In controlled liquid evaporation, as a very small amount of liquid is left, about a drop in size, the droplet is almost de-coupling and absorbs very little energy. Cool vessel walls at or below ambient room temperature with residual cooling or with ambient cooling resist elevated temperatures from being transferred to the sample residue if it does not absorb microwave energy directly or efficiently. In addition, active cooling of selected vessel surfaces; removal of molecular solvent molecules, addition of cool gasses and other manipulations resist losses of both elemental and molecular analytes.

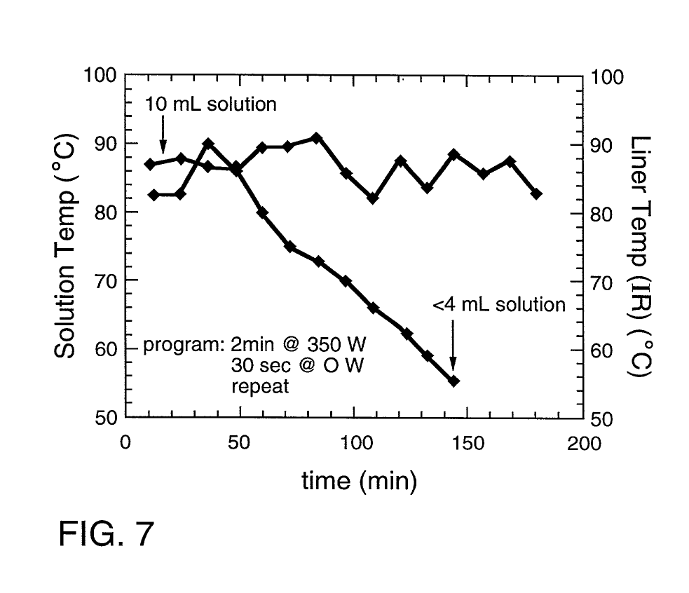

To demonstrate these manipulations, FIG. 7 shows the cooling of the solution in a microwave energy field as it evaporates and becomes relatively smaller in relation to the specific microwave wavelength. Here microwave energy at a frequency of 2450 MHz is applied. In general, in the present invention, it will be preferred to employ a microwave frequency of about 27 to 2450 MHz. FIG. 7 is a plot of temperature, as the size of the remaining acid becomes small as the sample and solvent approach dryness. In this particular case hydrochloric acid is the solvent, however, this effect is common to microwave absorbing solvents. As can be seen the temperature of the liquid as it approaches dryness lowers, not rises. This mechanism is opposite to that of convection and conduction as previously described herein. As the amount of solvent, hydrochloric acid in this case, becomes small and approaches dryness it eventually essentially uncouples and the droplet stops heating all together just before dryness.

This test employed 10 mL 10:% HCl solution in the vessel. Temperature of solution was measured after each 12 minute heating cycle. As the solution volume reduced to<4 mL, use of the thermocouple made accurate read temperature at lesser volumes difficult.

In these studies an apparatus was constructed to permit exploitation of the microwave evaporation mechanism and active cooling of the vapor phase.

Other variations in the process and related apparatus may include active cooling of selected vessel surfaces such as annular wall 122 (FIG. 5) by any suitable means disclosed herein or acceptable substitutes therefor. The gas phase may be removed from the vessels in a manner which will be discussed herein in order to facilitate cooling efficiency and permit further processing of the gas phase. The gas phase so removed may be replaced as by introduction of other gases. The addition of gases to the vapor phase, such as air, nitrogen and/or carbon dioxide for the purpose of cooling or residual solvent removal or shifting equilibrium, for example, is contemplated. Reagents may be added to the vessel interior and a vacuum may be drawn on the vessel interior or other means employed to assist with achieving the desired pressure.

In this experiment the solvent and gas phase were removed from the vessel by vacuum. The vessel was cooled by microwave transparent air on the top and sides of the vessel (<20° C. air) and the vaporized (SKIP?) acid and water removed in the gas stream. The trace element salts and molecules with low or no appreciable vapor pressure at low or laboratory temperature were not removed with the solvent. They remain in the solution volume and finally in the sample residue that is at a temperature of about ambient temperature. A comparative study of both hot-plate and microwave evaporation demonstrates the losses when convection and conduction are used in the traditional manner and how analytes are conserved in the microwave evaporation technique.

In the experiment 1 μg/g in 10% hydrochloric acid solutions were prepared with Be, Cr, V, Mn, Ni, Cu, Zn, As, Ag, Sn, Sb, Pb and Hg. Data in Tables 2 and 3 show confirming analytical evidence of the different effects of the two mechanisms. These studies compare standard hot-plates and pyrex glass beakers (Table 2) with the special microwave apparatus (Table 3) constructed of fluoropolymers with the ability to remove gas phases and cool vessel surfaces.

For Table 2, the hot-plate evaporation to dryness procedure involved 10 ppm solution being prepared by diluting commercially obtained ICP-MS elemental standards in a final matrix of 10% HCl. About 10 mL of this solution was placed in Pyrex beakers and evaporated down to dryness. Beakers were rinsed and diluted to 50 mL. Any residue remaining in beaker was redissolved in 6 mL of 50% HNO₃, rinsed, and diluted to 50 mL. The initial evaporation rinse and the residue rinse were analyzed individually and the results were added to determine the total jug remaining in each beaker after evaporation. The solutions were not permitted to boil in order to prevent mechanical losses from bubbles. The original solution contained 100 μg (10 mL×10 μg/mL). Three to four samples were done in each trial. Statistically significant losses occurred with As, Se, Sn, Sb, Hg. Losses were not controllable or reproducible. The complete recovery of non-volatile elements and compounds (Cu, Zn, Ni) show that the only losses were of volatile components of the solution.

TABLE 2

| | |
|---|---|
| Original Solution | Evaporated Solution |

| Element | Average | 95% C.I. | St. Dev | Average | 95% C.I. | St. Dev |
|---|---|---|---|---|---|---|
| Cr 50 | 108.65 | 2.68 | 1.08 | 107.68 | 3.24 | 2.037 |
| V 51 | 101.87 | 2.04 | 0.82 | 99.39 | 2.81 | 1.766 |
| Cr 52 | 100.77 | 1.04 | 0.42 | 103.79 | 3.65 | 2.295 |
| Mn 55 | 102.89 | 2.57 | 1.04 | 103.73 | 1.53 | 0.959 |
| Ni 60 | 102.02 | 0.35 | 0.14 | 104.20 | 1.52 | 0.955 |
| Ni 62 | 101.52 | 6.20 | 2.50 | 104.27 | 1.26 | 0.793 |
| Cu 63 | 101.61 | 2.36 | 0.95 | 103.22 | 1.41 | 0.889 |
| Zn 64 | 102.47 | 1.61 | 0.65 | 103.91 | 1.00 | 0.629 |
| Cu 65 | 100.36 | 3.16 | 1.27 | 101.62 | 0.58 | 0.366 |
| Zn 66 | 102.61 | 4.18 | 1.68 | 102.52 | 1.91 | 1.203 |
| As 75 | 99.70 | 1.22 | 0.49 | 79.13 | 8.61 | 5.417 |
| Se 78 | 107.50 | 7.58 | 3.05 | 87.06 | 3.54 | 2.225 |
| Se 82 | 97.65 | 4.47 | 1.80 | 86.75 | 6.48 | 4.077 |
| Sn 118 | 97.54 | 3.99 | 1.61 | 62.91 | 8.84 | 5.559 |
| Sn 120 | 99.59 | 4.66 | 1.88 | 63.19 | 9.03 | 5.682 |
| Sb 121 | 99.21 | 4.07 | 1.64 | 78.97 | 9.56 | 6.013 |
| Sb 123 | 98.61 | 1.98 | 0.80 | 79.22 | 10.00 | 6.289 |
| Hg 200 | 99.29 | 2.62 | 1.06 | 41.57 | 39.26 | 24.69 |
| Hg 202 | 96.79 | 3.96 | 1.59 | 45.29 | 36.87 | 23.19 |

The abbreviation “C.I.” refers to the confidence interest and “St. Dev.” refers to standard deviation.

Table 3 shows the results of the system of the present invention involving microwave evaporation of standard solution to dryness using special vessel apparatus with cooling and vapor extraction capability. A 1 ppm solution was prepared by diluting commercial multi-element ICP-MS standards in a final matrix of 10% HCl. An 8 mL quantity of this solution was placed in fluoropolymer vessels and evaporated to dryness. After 90 minutes of cycling through heating program, the samples were dry with some condensed solvent on the cool wall and cap of the vessel. The vessels were rinsed, diluted to 50 mL, and analyzed by ICP-MS. The original solution contained 8 μg (8 mL×1 μg/mL). Three to four samples were employed in each trial.

TABLE 3

| | Original Solution | | | Evaporated Solution | | |
|---|---|---|---|---|---|---|
| Element | Mean | 95% C.I. | St. Dev. | Mean μg | 95% C.I. | St. Dev. |
| Be 9 | 7.27 | 0.13 | 0.08 | 7.60 | 0.22 | 0.14 |
| Cr 50 | 7.43 | 0.13 | 0.08 | 7.67 | 0.53 | 0.33 |
| V 51 | 8.03 | 0.08 | 0.05 | 7.38 | 0.82 | 0.52 |
| Cr 52 | 7.65 | 0.02 | 0.01 | 7.93 | 0.06 | 0.04 |
| Mn 55 | 7.65 | 0.07 | 0.05 | 8.06 | 0.08 | 0.05 |
| Ni 60 | 7.53 | 0.08 | 0.05 | 8.04 | 0.06 | 0.04 |
| Ni 62 | 7.51 | 0.14 | 0.09 | 7.98 | 0.13 | 0.08 |
| Cu 63 | 7.62 | 0.07 | 0.04 | 8.04 | 0.17 | 0.11 |
| Zn 64 | 7.82 | 0.04 | 0.03 | 8.28 | 0.25 | 0.16 |
| Cu 65 | 7.60 | 0.01 | 0.00 | 8.12 | 0.12 | 0.07 |
| Zn 66 | 7.64 | 0.13 | 0.08 | 8.24 | 0.22 | 0.14 |
| As 75 | 7.76 | 0.08 | 0.05 | 7.34 | 0.87 | 0.54 |
| Ag 107 | 7.74 | 0.05 | 0.03 | 6.47 | 2.92 | 1.84 |
| Ag 109 | 7.72 | 0.06 | 0.04 | 6.45 | 2.97 | 1.87 |
| Sn 118 | 7.60 | 0.18 | 0.11 | 7.97 | 0.11 | 0.07 |
| Sn 120 | 7.64 | 0.13 | 0.08 | 7.95 | 0.19 | 0.12 |
| Sb 121 | 7.64 | 0.24 | 0.15 | 8.00 | 0.15 | 0.10 |
| Sb 123 | 7.63 | 0.11 | 0.07 | 8.01 | 0.12 | 0.07 |
| Pb 206 | 7.82 | 0.17 | 0.10 | 8.10 | 0.11 | 0.07 |
| Pb 208 | 7.81 | 0.12 | 0.08 | 8.13 | 0.07 | 0.04 |

These results demonstrate that analytes are completely recovered using the microwave evaporation. In this study dryness was achieved in approximately 90 minutes. This heating program can be optimized to achieve similar results in shorter times with higher powers and optimized conditions. No statistically significant loss was observed from the microwave evaporation because of cooling of final phase of evaporation.

To validate the accuracy of this evaporation method standard reference materials were decomposed in 10 mL of nitric acid. Prior to evaporation 2 mL of hydrochloric acid was used to complex Ag, Sb and other elements that are insoluble in nitric acid solution. Evaporation was carried out in the same fluoropolymer vessel and access for removal of solvent molecules and cooling. The results are shown in Table 4 wherein “M/W” at the head of a column relates to individual samples.

TABLE 4

| NET RESULTS FOR | | | | | | | | | | |
|---|---|---|---|---|---|---|---|---|---|---|
| MICROWAVE EVAPORATED SAMPLES | | | | | | | | | | |
| SAMPLE | | MW | MW | MW | MW | EV4 | | CERTIFIED | | |
| Element | Isotop | μg/g | μg/g | μg/g | μg/g | Mean | 95% | LEVEL | MEAN | 95% C.I. |
| V | 51 | 4.66 | 4.36 | 4.58 | 4.77 | 4.59 | 0.27 | V | 4.68 | 0.15 |
| Cr | 52 | 1.78 | 1.76 | 1.75 | 1.74 | 1.76 | 0.03 | Cr | 1.43 | 0.46 |
| Mn | 55 | 11.54 | 11.02 | 11.67 | 11.74 | 11.49 | 0.52 | Mn | 12.3 | 1.5 |
| Co | 59 | 0.49 | 0.49 | 0.50 | 0.48 | 0.49 | 0.02 | Co | 0.57 | 0.11 |
| Ni | 60 | 3.16 | 1.63 | 2.10 | 2.25 | 2.29 | 1.02 | Ni | 2.25 | 0.44 |
| Cu | 63 | 62.56 | 58.58 | 62.55 | 61.67 | 61.34 | 3.00 | Cu | 66.3 | 4.3 |
| Cu | 65 | 63.80 | 58.49 | 62.49 | 63.93 | 62.18 | 4.04 | | | |
| As | 75 | 14.97 | 12.35 | 15.06 | 12.91 | 13.82 | 2.22 | As | 14.0 | 1.2 |
| Ag | 107 | 1.50 | 1.55 | 1.53 | 1.55 | 1.53 | 0.04 | Ag | 1.68 | 0.15 |
| Ag | 109 | 1.53 | 1.54 | 1.55 | 1.55 | 1.54 | 0.02 | | | |
| Cd | 111 | 4.14 | 3.94 | 4.25 | 4.39 | 4.18 | 0.30 | Cd | 4.15 | 0.38 |
| Cd | 114 | 4.19 | 3.90 | 4.20 | 4.23 | 4.13 | 0.24 | | | |
| Pb | 206 | 0.39 | 0.28 | 0.40 | 0.45 | 0.38 | 0.11 | Pb | 0.371 | 0.014 |
| Pb | 208 | 0.39 | 0.32 | 0.39 | 0.43 | 0.38 | 0.07 | | | |

These data demonstrate the retention of trace elements that would normally be lost in evaporation after decomposition during the solvent modification steps in most procedures. Here they are retained and give the certified values. Chromium and arsenic are two elements that are lost frequently from chloride containing solutions and are notorious for this behavior. Here they and all the elements are quantitatively manipulated using the methods described.

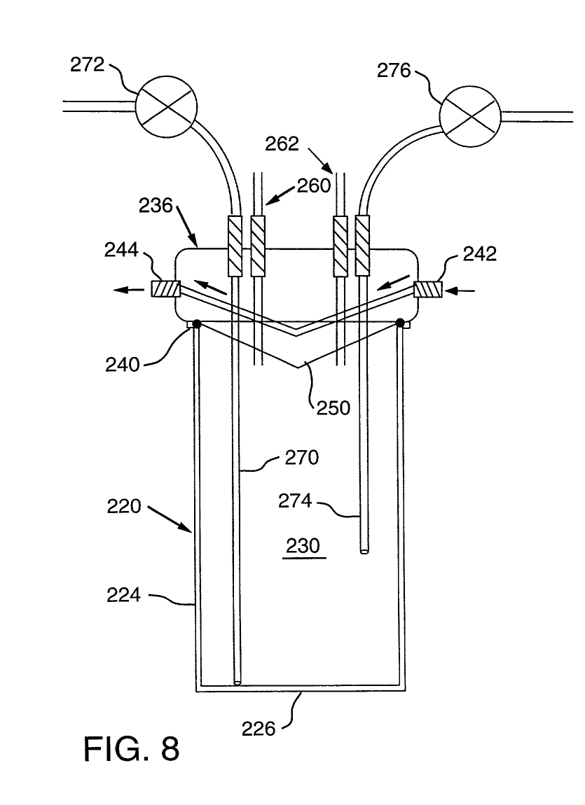

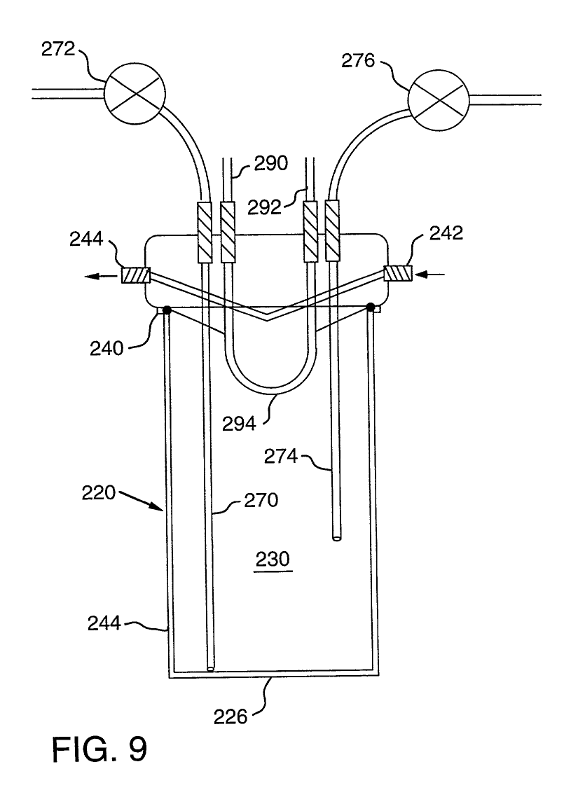

Referring to FIGS. 8 and 9, modified forms of vessels and additional details regarding material delivery and removal options, as well as cooling units will be considered. In FIG. 8, a vessel 220 which is at least partially transparent to microwave energy and may be generally cylindrical in shape has an annular sidewall 224 and an integrally formed base 226. A processing chamber 230 is provided. In the form shown, the chamber 230 is a single chamber. Sealingly secured to the vessel 220 which may be composed of a fluoropolymer is a cooling unit 236. A suitable annular seal 240 such as a fluoropolymer coated O-ring seal is provided. The cooling unit has a pair of ports 242, 244 one of which will introduce coolant into the cooling unit 236 and the other of which will withdraw the coolant, such as having entry through 242 and exit through 244. It will be appreciated that in this embodiment, the cooling unit 236 has a downwardly projecting portion 250 which enhances the cooling action on the gases contained in the upper portion of chamber 230.

Also contemplated by this embodiment of the present invention are a pair of gas, air or vacuum ports 260, 262 which are in communication with the chamber 230. These may be employed to exhaust the gases created during the processing of the sample or to draw a vacuum on the chamber 230 or to introduce air, nitrogen or carbon dioxide, for example, into the system. The process may be continuous with a reagent removal tube 270 passing through the cooling unit 236 and being operatively associated with a pump 272 which facilitates removal of reagent. Also, reagents may be introduced into the chamber 230 through tube 274 under the influence of pump 276.

With respect to FIG. 9, the apparatus may be essentially as shown in FIG. 8 except for providing additional cooling capacity through the flow of coolant in tube 294 with tube portions 290, 292 serving as entry and exit passageways for a coolant, such as liquid nitrogen. In this embodiment, tubes 290 and 292 have a generally U-shape lower portion 294 which projects downwardly into processing chamber 230.

It will be appreciated that the present invention may be employed advantageously with a wide variety of materials and end uses. The following examples will illustrate some advantageous uses. Among the specific end uses for which the sample preparation, method and apparatus of the present invention may be employed are microwave assisted decomposition, synthesis, derivatization and/or extraction or leaching, chemical analysis or microwave distillation purification. The invention may be employed to perform mineral acid decompositions while cooling the acid vapor to reduce the temperature and responsively the pressure of the decomposition system. Also, organic extraction with organic solvents may be performed while cooling the gas phase to reduce the pressure of the overall reaction.

For purposes of clarity of illustration and disclosure, FIGS. 5 and 6 have illustrated certain features, such as the presence of two compartments. FIGS. 8 and 9 have illustrated means for introducing or removing materials into or out of the vessel and cooling means having certain preferred features. It will be appreciated by those skilled in the art that individual features from FIGS. 5 or 6 may be employed with individual features of FIGS. 8 or 9, if desired.

The methods and associated apparatus of the present invention are applicable to a wide range of technical and industrial fields. For example, in addition to other usage which will be apparent to those skilled in the art, uses in analytical chemistry, environmental chemistry, industrial chemistry, food chemistry and industrial processing and engineering of associated microwave apparatus are potential uses.

The invention may be employed to perform organic or inorganic synthesis with solvents while cooling the gas phase to reduce the pressure during synthesis.

The invention may also be employed to perform hydrolysis on a protein with a solvent mixture including hydrochloric acid and cooling the gas phase to effect a reduction in pressure during hydrolysis. Another use is drying to condense components of the vapor phase.

In some instances, the gas phase may be cooled to resist temperature damage to the material out of which the inner liner or outer casings are made, such as polyetherimide, for example. The invention may also be employed with azeatropes, as well as aqueous materials.

Uses in environmental, biological, medical and industrial fields will be readily apparent to those skilled in the art.

The invention may be employed with all types of microwave systems including, for example, cavity-type microwave systems, focused microwave systems, flow and stop flow microwave systems, and antenna transmitted microwave cavities.

With respect to the liquid temperature, if desired one may operate at a higher liquid temperature with no increase in vessel internal pressure or at similar liquid temperatures with a decrease in pressure.

The invention further facilitates resisting undesired escape of the volatile elements, molecules, and compound losses when opening vessels to the atmosphere and condensing of these from the gas phase.

Whereas particular embodiments of the invention have been described herein for purpose of illustration, it will be evident to those skilled in the art that numerous variations of the details may be made without departing from the invention as defined in the appended claims.

1. A method of microwave assisted chemical analysis comprising,
providing a sample within a vessel,
heating said sample by microwave energy to volatilize at least a portion of said sample to establish a gas phase,
cooling said gas phase while continuing to heat said sample by said microwave energy, and
analyzing the unvolatilized portion of said sample to determine the composition of said unvolatilized portion.

2. The method of claim 1 including
employing a said sample containing silicon, and
said unvolatilized portion including trace elements contained in said silicon containing sample.

3. The method of claim 2 including
determining the identity and quantity of at least some of said trace elements.

4. The method of claim 1 including
performing said process in a closed said vessel.

5. The method of claim 1 including
withdrawing at least a portion of said gas phase from said vessel.

6. The method of claim 1 including
employing as said vessel a vessel which has portions which are transparent to microwave energy.

7. The method of claim 2 including
employing polycrystalline silicon as said sample.

8. The method of claim 2 including
employing a vessel with at least two compartments in communication with each other,
introducing a silicon containing sample and a first acid into a first said compartment, and
introducing a second acid into a second said compartment.

9. The method of claim 8 including
employing nitric acid as said first acid, and
employing hydrofluoric acid as said second acid.

10. The method of claim 9 including
distilling said hydrofluoric acid into said first compartment, and
distilling SiF₄ into said second compartment.

11. The method of claim 1 including employing as said microwave energy energy of a frequency of about 27 to 2450 megahertz.

12. The method of claim 11 including
employing a said vessel composed of a fluoropolymer.

13. The method of claim 1 including
said vessel having a unitary chamber.

14. The method of claim 1 including
during said process introducing additional sample into said vessel.

15. The method claim 14 including
said process being a continuous process.

16. The method of claim 1 including
employing a liquid as said sample.

17. The method of claim 1 including
during said process introducing additional said sample into said vessel.

18. The method of claim 17 including
during said process withdrawing at least a portion of said gas phase from said vessel.

19. The method of claim 2 including
effecting substantially complete retention in said unvolatilized portion of all of said trace elements.

20. A method of microwave assisted chemical purification comprising
providing a sample within a vessel,
heating said sample by microwave energy to volatilize at least a portion of said sample to establish a gas phase,
cooling said gas phase while continuing to heat said sample by said microwave energy, and
purifying at least a portion of said gas phase to establish a purified portion of said sample.

21. The method of claim 20 including
employing a said sample containing silicon,
converting said sample into said gas phase and unvolatilized portions, and
said unvolatilized portions being trace elements originally contained in said silicon containing sample.

22. The method of claim 21 including
performing said process in a closed said vessel.

23. The method of claim 21 including
withdrawing at least a portion of said gas phase from said vessel.

24. The method of claim 21 including
employing as said vessel a vessel which has portions which are transparent to microwave energy.

25. The method of claim 21 including
employing polycrystalline silicon as said sample.

26. The method of claim 21 including
employing a vessel with at least two compartments in communication with each other,
introducing a silicon containing sample and a first acid into a first said compartment, and
introducing a second acid into a second said compartment.

27. The method of claim 26 including
employing nitric acid as said first acid, and
employing hydrofluoric acid as said second acid.

28. The method of claim 27 including
distilling said hydrofluoric acid into said first compartment, and
distilling SiF₄ into said second compartment.

29. The method of claim 20 including
employing as said microwave energy energy of a frequency of about 27 to 2450 megahertz.

30. The method of claim 29 including
employing a said vessel composed of a fluoropolymer.

31. The method of claim 20 including
said vessel having a unitary chamber.

32. The method of claim 1 including
during said process introducing additional sample into said vessel.

33. The method of claim 20 including
employing a liquid as said sample.

34. The method of claim 20 including
during said process withdrawing at least a portion of said gas phase from said vessel.

35. The method of claim 21 including
employing a said sample containing silicon, and
said unvolatilized elements being trace elements contained in said silicon containing sample.

36. Apparatus for a microwave assisted chemical analysis comprising
a vessel transparent to microwaves for receiving a sample containing at least one other material,
said vessel having a space above the region wherein said sample will be present for receiving a gas phase,
cooling means for positively cooling said gas phase to reduce the temperature of said gas phase without effecting substantial cooling of said liquid phase, and
means for obtaining access to the portion of said sample which has not been converted into said gas phase.

37. The apparatus of claim 36 including
means for withdrawing at least a portion of said gas phase from said vessel.

38. The apparatus of claim 37 including
means for introducing additional said sample into said vessel.

39. The apparatus of claim 36 including
said vessel being structured to receive said sample in a lower portion thereof, and
said cooling means being disposed in an upper portion of said vessel.

40. The apparatus of claim 36 including
said vessel having at least two compartments in communication with each other.

41. The apparatus of claim 36 including
means for introducing into said vessel at least one material selected from the group consisting of air, nitrogen and carbon dioxide.

42. The apparatus of claim 39 including
said cooling means having a portion extending into said vessel.
